(19) United States
(12) Patent Application Publication
(10) Pub. No.: US 2018/0271639 A1
(43) Pub. Date: Sep. 27, 2018

(54) MEDICAL DEVICES FOR CONTROLLED DRUG RELEASE

(71) Applicant: COOK MEDICAL TECHNOLOGIES LLC, BLOOMINGTON, IN (US)

(72) Inventors: Ralf Spindler, Bloomington, IN (US); Georgios Hilas, Bloomington, IN (US)

(73) Assignee: COOK MEDICAL TECHNOLOGIES LLC, BLOOMINGTON, IN (US)

(21) Appl. No.: 15/927,299

(22) Filed: Mar. 21, 2018

Related U.S. Application Data
(60) Provisional application No. 62/474,935, filed on Mar. 22, 2017.

(51) Int. Cl. A61F 2/07; D01D 5/00; A61L 27/56

(52) U.S. Cl. CPC A61F 2/07; D04H 1/728; A61L 27/56; D01D 5/003

(57) ABSTRACT

A medical device for controlling a release of bioactive agents to a body of patient. The medical device includes a support structure and a coating disposed at least partially covering the support structure. The coating includes a biodegradable outer and inner layer. First beads are disposed between the layers. A therapeutically effective amount of a first bioactive agent is disposed within pores of the first beads. Second beads are disposed between the support structure and the inner layer. A therapeutically effective amount of a second bioactive agent is disposed within the pores of the second beads. The first beads are releasable in response to at least partial biodegradation of the outer layer, and the second beads are releasable after the first beads in response to at least partial biodegradation of the inner layer. The coating may include a nonwoven matrix.

### PRIORITY CLAIM

This invention claims the benefit of priority of U.S. Provisional Application Ser. No. 62/474,935, entitled “Medical Devices for Controlled Drug Release,” filed Mar. 22, 2017, the disclosure of which is hereby incorporated by reference in its entirety.

### BACKGROUND

The present disclosure relates generally to medical devices, and particularly, to endoluminal prostheses, stent-grafts, or stents with a coating for controlled drug release and methods for the manufacture and use of the same for repair of damaged vessels, ducts, or other physiological pathways.

Various interventions have been provided for weakened, aneurysmal, dissected or ruptured vessels, including surgical interventions and endovascular interventions. Endovascular interventions generally include inserting an endoluminal device or prosthesis such as a stent or stent graft into the damaged or diseased body lumen to provide support for the lumen, and to exclude damaged portions thereof. Such prosthetic devices are typically positioned at the point of treatment or target site by navigation through the vessel, and possibly other connected branch vessels, until the point of treatment is reached. This navigation may require the device to be able to move axially through the vessel(s) prior to deployment, while still maintaining the ability to exert an outward force on the interior wall once deployed.

In the field of aortic interventions, endoluminal devices are placed in vessels to address and correct diseased tissue resulting from atherosclerotic plaques, aneurysm or weakening of body vessel walls, and arterial dissection. In the case of atherosclerosis, plaque buildup results in narrowing of the vessel, which may lead to reduced or blocked blood flow within the body vessel. Endoluminal device for atherosclerosis acts to radially expand the narrowed area of the body vessel to restore normal blood flow. In the case of an aneurysm, a weakening of the body vessel wall results in ballooning of the body vessel, which can eventually lead to rupture and subsequent blood loss. In some cases, the aneurysmal sac may include plaque. Endoluminal device for aneurysms acts to seal off the weakened area of the body vessel to reduce the likelihood of the body vessel rupture. In the case of arterial dissection, a section of the innermost layer of the arterial wall is torn or damaged, allowing blood to enter false lumen divided by the flap between the inner and outer layers of the body vessel. The growth of the false lumen may eventually lead to complete occlusion of the actual artery lumen. An endoluminal device for dissection healing would reappose the dissection flap against the body vessel wall to close it off and restore blood flow through the true lumen.

After such endoluminal device placement for the various aortic interventions, different response may occur including thrombosis, inflammation, smooth muscle cell migration and proliferation, extracellular matrix production and reabsorption. These various body responses may result in re-stenosis of the stent lumen, leading to eventual occlusion of the body vessel and/or formation of emboli along the inner surface of the covered stent material. The emboli may separate and result in vessel blockages downstream.

### SUMMARY

Medical devices are disclosed herein for controlling a release of bioactive agent to a body of patient. In one example, a medical device includes a support structure and a coating disposed at least partially covering the support structure. The coating includes one or more layers comprising at least one of a biodegradable material and a biostable material. Beads may be associated with at least one of the coating layers. A therapeutically effective amount of a bioactive agent is associated with the beads. Beads are releasable in response to at least partial biodegradation of the biodegradable material found in the respective layers. In one example, first beads are releasable in response to at least partial biodegradation of an outer layer, and second beads are releasable after the first beads in response to at least partial biodegradation of an inner layer. In one example, beads are releasable in response to at least partial biodegradation of an outer layer and at least partial biodegradation of an inner layer. Other systems, methods, features and advantages of the invention will be, or will become, apparent to one with skill in the art upon examination of the following figures and detailed description. It is intended that all such additional systems, methods, features and advantages be within the scope of the invention, and be encompassed by the following claims.

### BRIEF DESCRIPTION OF THE DRAWINGS

The invention can be better understood with reference to the following drawings and description. The components in the figures are not necessarily to scale, emphasis instead being placed upon illustrating the principles of the invention. Moreover, in the figures, like referenced numerals designate corresponding parts throughout the different views.

FIG. 1 is a perspective view of an example of a medical device.

FIG. 2 depicts the medical device in FIG. 1 implanted within a body vessel, shown as the aortic system.

FIG. 3 depicts another example of a medical device.

FIG. 4 is a magnified cross-sectional view of one example of a partial coating for use with a medical device.

FIG. 5 depicts a body of a bead.

FIGS. 6A-6C are magnified sectional views of a single pore of different configurations of the bead in FIG. 5.

FIG. 7 is a magnified cross-sectional view of another example of a partial coating for use with a medical device.

FIGS. 8A-8D depict another example of a partial coating for use with a medical device at four distinctive and sequential times at or after implantation of the medical device into the body vessel.

FIGS. 9A-9D depict another example of a partial coating for use with a medical device at four distinctive and sequential times at or after implantation of the medical device into the body vessel.

FIGS. 10A-10C depict another example of a partial coating for use with a medical device at three distinctive and sequential times at or after implantation of the medical device into the body vessel.

### DETAILED DESCRIPTION OF THE PREFERRED EMBODIMENTS

Medical devices for implantation within a human or animal body for repair of damaged vessels, ducts, or other physiological pathways are provided. The medical devices may have coating and/or graft materials with one or more bioactive agents. The bioactive agents may be proportionally or disproportionally distributed along the coating or graft material. The coating/graft material and bioactive agent configurations may provide the ability to control the bioactive agent release at a desired rate over a single or plurality predetermined time periods. This release controlled ability may provide treatment of different conditions or diseases at different times. For example, an anti-plaque, anti-thrombosis agent, and/or another agent may be initially delivered for therapeutic effects within hours of implantation, whereas a pro-coagulate and/or another agent may be released later, for example, within weeks, for therapeutic effects to withstand endoleaks. Release of the beads may also be controlled with biodegradable layers have different configurations. The coating/graft material may have desirable properties of the coating/graft material at different times, such as a less porous structure initially, and more and/or greater porous later.

In the present application, the term “proximal end” is used when referring to that end of a medical device closest to the heart after placement in the human body of the patient, and may also be referred to as inflow end (the end that receives fluid first), and the term “distal end” is used when referring to that end opposite the proximal end, or the one farther from the heart after its placement, and may also be referred to as the outflow end (that end from which fluid exits).

The term “biodegradable” is used herein to refer to materials selected to dissipate upon implantation within a body, independent of which mechanisms by which dissipation can occur, such as dissolution, degradation, absorption and excretion, and these terms may be used interchangeable throughout the description. The actual choice of which type of materials to use may readily be made by one ordinarily skilled in the art. Such materials are often referred to by different terms in the art, including “bioresorbable,” “bioabsorbable,” or “biodegradable,” depending upon the mechanism by which the material dissipates.

The term “bioabsorbable” is used herein to refer to materials with a detectable loss of mass that takes place, even if it occurs at a much later stage than the molecular weight reduction. A bioabsorbable polymer is degraded into water soluble, low molecular weight compounds such as monomer units, which are then absorbed by surrounding body fluid. The term “bioresorbable” is used herein to refer to materials that can be broken down and absorbed by the body and thus need not be removed manually. For the purposes of this application, unless otherwise specified, the term “biodegradable” includes materials that are “bioresorbable,” and “bioabsorbable.” The prefix “bio” indicates that the erosion occurs under physiological and biological conditions, as opposed to other erosion processes, caused by, for example, high temperature, strong acids or bases, UV light or weather conditions. As used herein, “biodegradable material” includes materials, such as a polymer or copolymer, which are absorbed by the body, as well as materials that degrade and dissipate without absorption into the body. As used herein, “biodegradable polymer” refers to a polymer or copolymer, which dissipates upon implantation within the body. A large number of different types of materials are known in the art which may be inserted within the body and later dissipate.

“Biostable” material refers to a material, such as a polymer or copolymer, which remains in the body without substantial biodegradation.

Medical device may be any device that is introduced temporarily or permanently into the body for the prophylaxis or therapy of a medical condition. For example, such medical devices may include, but are not limited to: endovascular grafts, stents, stent grafts, bifurcated stent grafts or assembly of a multicomponent prosthesis, balloon catheters, meshes, vascular grafts, stent-graft composites, filters (for example, vena cava filters), vascular implants, tissue scaffolds, myocardial plugs, valves (for example, venous valves), various types of dressings, endoluminal prostheses, vascular supports, or other known biocompatible devices.

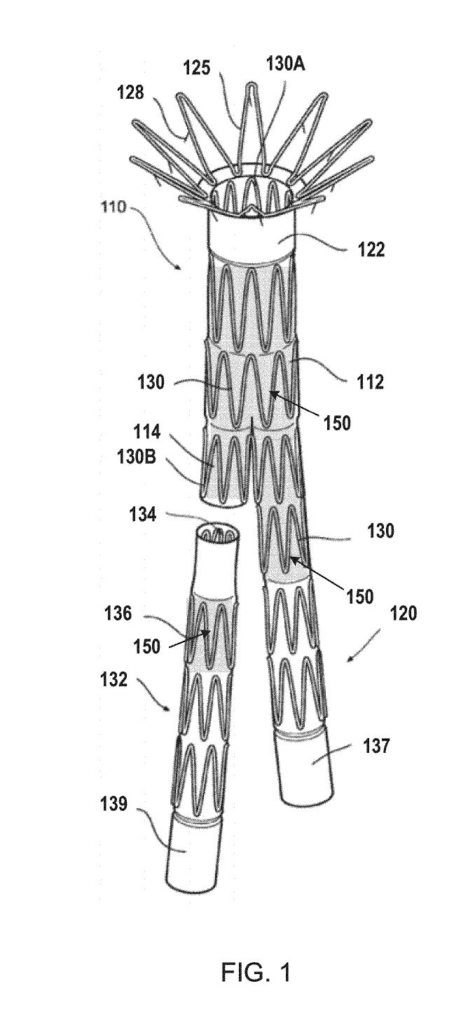

Now looking more closely at the drawings, FIGS. 1-3 depict one example of a medical device, including a tubular graft support body and/or one or more support frame structures disposed along the graft support body. The graft body may be cylindrical along the entire length. It is contemplated the graft body may have segments that are tapered. In one example, the prosthesis is suitable for placement into an aorta and engaging against the aorta.

FIG. 1 shows the medical device as a stent graft 110 having leg portions, although the prosthesis may be configured without legs or branches. The stent graft 110 is shown having a generally inverted Y-shaped configuration having a body portion 112, a shorter leg 114 and a longer leg 120. In other examples, the legs 114, 120 may be substantially the same size. In other examples, the legs 114 and/or 120 may be integrally formed with the body portion 112, rather than assembled in-vivo such as shown in FIG. 1. The support body of the stent graft is constructed from a graft material, such as one of the graft materials described below. At a proximal end 122 of the stent graft 110 may be a first stent 125 which extends beyond the proximal end 122 and has distally extending anchoring members 128. The anchoring members 128 may include a barb or element with sharpened tip that is separately formed and securely attached to the first stent 125. In other examples, the anchoring members 128 include barb-like structures formed integrally with the first stent 125 during formation of the first stent. When radially expanded, the first stent 125 is configured to inhibit movement or migration of the stent graft by securely engaging the inner wall of the body vessel.

The stent graft 110 may have one or more of body stents 130 disposed along the body portion 112 distal to the first stent 125. The body stents 130 may be disposed along the inner surface, the exterior surface or a combination of both, of the tubular material of the body portion 112. When the body stent is placed nearest the proximal end 122 (or proximal body stent 130A), the body stent 130A may be disposed along inside the tubular material of the body portion 112 so that the outside presents a smooth surface which in use engages against the inner wall of the body vessel into which it is deployed to provide a barrier to endoleaks. The body stent nearest the distal end (distal body stent 130B) of the shorter leg or the longer leg may be outside the tubular material so that the inside presents a smooth surface which in use engages against the outside of the proximal end of a leg extension stent graft 132. Between the distal body stent 130B and the proximal body stent 130A, the rest of the stents 130 may be arranged on the outside of the tubular material to provide an unobstructed surface that presents minimal restriction to the flow of blood through the stent graft and present minimal sites for the growth of thromboses within the stent graft. The body stents 130 may also be disposed along the longer leg 120.

Extension leg stent graft 132 is adapted for fitting into the distal end opening 133 of the shorter leg 114, such as shown in FIG. 2. The extension stent graft 132 is constructed from a tubular any one of the graft materials described herein. The extension stent graft 132 may include a proximal terminal internal stent 134 to provide a bare smooth exterior surface for sealing with the inner surface of the shorter leg 114 when the proximal terminal internal stent 134 is radially expanded within the shorter leg 114. The extension stent graft 132 may include a plurality of external intermediate stents 136 to provide an unobstructed surface that presents minimal restriction to the flow of blood through the stent graft and present minimal sites for the growth of thromboses within the leg stent graft. The exterior surface of the distal end 137 of the longer leg 120 is adapted for sealing against the wall of the branch body vessel, such as one of the iliac arteries, and the distal end 139 of the extension leg stent graft 132 is adapted for sealing against the wall of the other branch vessel, such as the other iliac artery. Terminal internal distal stents (not shown) may be arranged to provide a bare smooth exterior surface at the distal ends for sealing with the body vessel. In another example, when the legs 114, 120 are omitted from the body portion 112 to define a tubular body, the distal end of the tubular body without legs may include an internal body stent to provide a bare exterior surface.

At least a portion of the graft material of the body portion 112, the shorter leg 114, the longer leg 120, or any combination thereof, may include a coating 150 (shown by the shading along the respective portions). The entire length of the graft materials may have the coating. In another example, less than the entire length of the graft material may have the coating. As will be described below, the coating 150 may be configured to release therapeutic agents into the body vessel to promote healing. The coating 150 may be further configured to release therapeutic agents in a time-controlled manner. The coating 150 may be further configured to release different therapeutic bioactive agents in a time-controlled manner. In one application, the bioactive agents may be beneficial for aneurysmic healing and to inhibit endoleaks, among other benefits as will be described, such as non-limiting examples of facilitating cellular migration (endothelialization) or reducing other complications, for example, thrombosis. In some examples, it is contemplated that the coating formed may be a substitute for the graft body. That is, the coating is configured as the support structure.

FIG. 2 shows the stent graft 110 deployed within an aorta 200 with an aneurysm 202. The aneurysm 202 is a ballooning of the aorta 200 and is shown between the renal arteries 204 and the iliac arteries 206. The stent graft 110 is deployed into the aorta 200 to span the aneurysm 202 and to allow blood flow from the aorta 200 to the two iliac arteries 206. At least a portion of the stent graft 110 that spans the aneurysm 202 may include the coating 150. The proximal end portion 122 (see FIG. 1) of the stent graft 110 which has the stent on the inside bears against the wall of the aorta 200 in the region above the aneurysm 202 and below the renal arteries 204 so that a good seal is obtained. The exposed first stent 125 (see FIG. 1) extending beyond the proximal end portion 122 extends over the entrances to the renal arteries 204, and the wire of the stent is thinner than the renal ostium so that occlusion does not occur. The distal end 137 of the longer leg 120 of the stent graft 110 seals against the wall of one of the iliac arteries 206, and the distal end 139 of the extension leg stent graft 132 bears against the wall of the other iliac artery 206.

In use, the stent graft 110 is adapted for fitting into the aorta such that the proximal end portion 122 is just distal of the renal arteries and the exposed first stent 125 extends up to or over the renal arteries. The longer leg 120 extends down one of the iliac arteries and the shorter leg 114 terminates in the aorta just short of the other iliac artery. The extension stent graft 132 when deployed inside the distal end 133 extends down the other iliac artery.

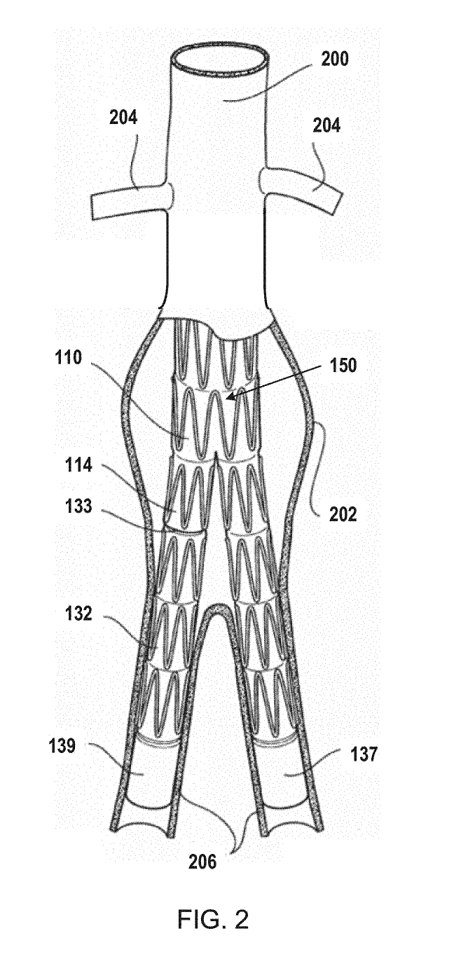

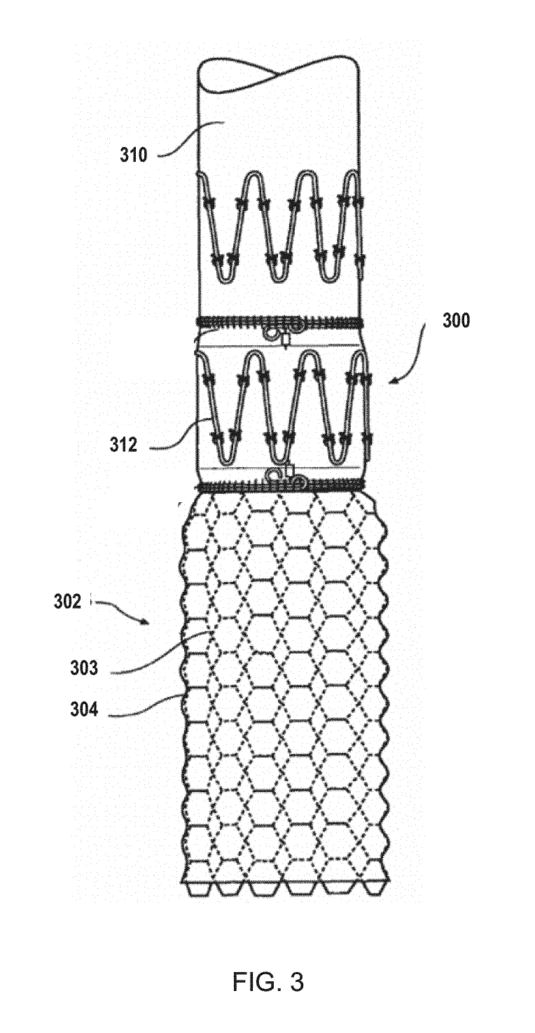

FIG. 3 illustrates another example of the medical device, referred to now as stent graft 300, without leg portions. The stent graft 300 may include any one or more of the features described above with respect to stent graft 110. Stent graft 300 further includes a support body structure 302 with a plurality of stent elements 303 covered with a cover or coating of graft material 304, such as a covered stent. The graft material 304 may be coated or extruded along the support body structure 302. In one example, the support body structure 302 includes a balloon expandable stent or balloon expanded covered stent. The support body structure 302 may comprise the entire length of the stent graft 300 or a portion of the stent graft 300, as shown. For example, the support body structure 302 is shown extending distal to the body portion 310, where the support body structure 302 is inserted within the distal end 312 of the body portion. The support body structure 302 may include a mesh of a shape memory metal such as nitinol or stainless steel. In another example, the support body structure may include other stent architecture design configurations of various annular rings interconnected to one another with longitudinal connector members. For example, the support body structure 302 may also be an interconnected stent element configuration cut from a cannula of shape memory metal such as nitinol or stainless steel. The medical device may comprise entirely of a covered stent configuration.

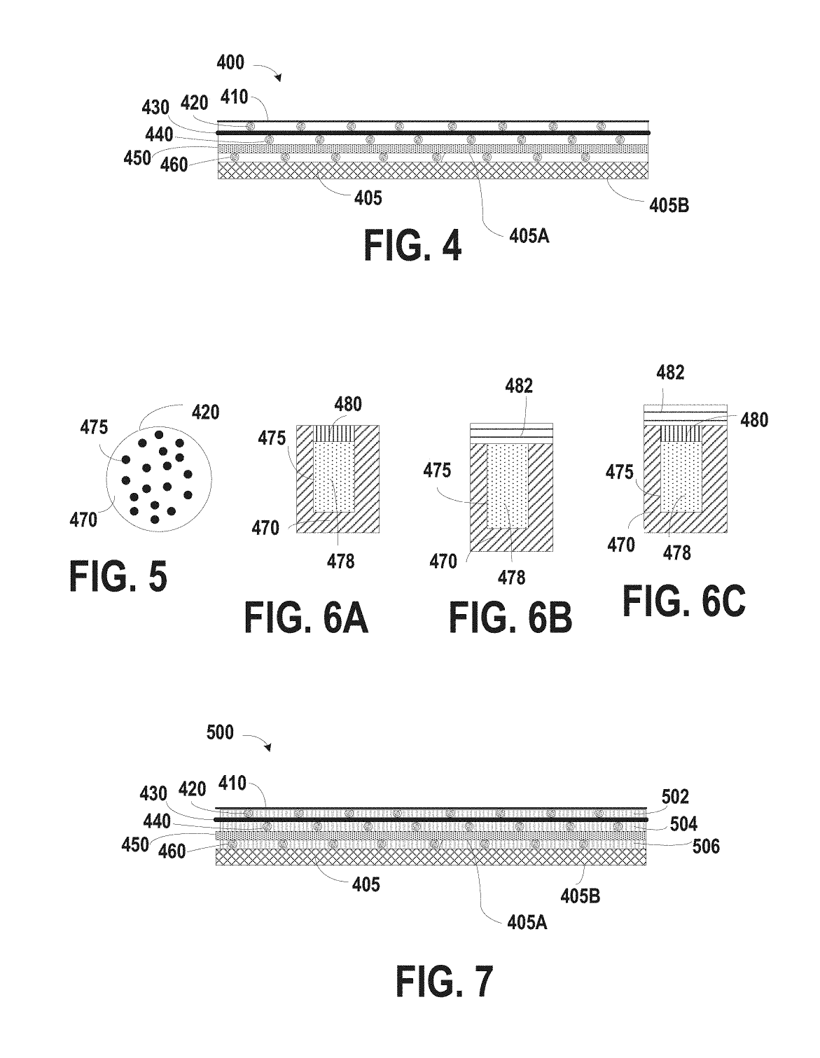

The figures illustrate examples of different coating configurations for a medical device, such as any one of the stent grafts disclosed herein. FIG. 4 illustrates a first coating 400 disposed along an outer surface 402 of a support structure 405, such as a graft material and/or a stent frame structure. The coating 400 includes one or more biodegradable layers and a plurality of beads comprising a bioactive agent. For example, the coating 400 is shown including an outer layer 410 comprising a biodegradable material. A plurality of first beads 420 comprising a bioactive agent form a layer disposed between the outer layer 410 and the support structure 405. Throughout the figures, the bioactive beads are shown schematically larger than what the beads would be at a microscopic level.

The coating 400 may include an inner layer 430 comprising a biodegradable material coaxially disposed inward relative to the outer layer 410. The layer of first beads 420 are sandwiched between the outer layer 410 and the inner layer 430. A plurality of second beads 440 comprising a bioactive agent form a second layer disposed between the inner layer 430 and the support structure 405. As shown, the coating 400 may include additional alternating biodegradable inner layers and bioactive bead layers disposed between the inner layer 430 and the support structure 405. For example, a second biodegradable inner layer 450 is coaxially disposed inward relative to the other biodegradable layers. The second layer of second beads 440 are shown sandwiched between the first inner layer 430 and the second inner layer 450 comprising a biodegradable material. A plurality of bioactive beads 460 form a third layer disposed between the second biodegradable inner layer 450 and the support structure 405. The third bioactive beads 460 may comprise any one of the agents described herein.

As described below, the medical device may be used as a bioactive delivery device having biodegrading layers after discrete periods of time. The biodegradable layers may be formed from the same materials or different materials. The biodegradable layers may be formed from the same materials having different characteristics such as wall thickness or porosity or different materials having different characteristics such as wall thickness or porosity.

Any suitable biodegradable material may be used for the layers, and a skilled artisan will be able to select one or more suitable biodegradable materials for use in a particular medical device based on various considerations, including any desired size and/or configuration of the medical device, any desired directional release of the bioactive in the medical device, and any desired degradation timeline for the medical device. Examples of suitable biodegradable materials for use in the outer and first and second inner layers of medical devices according to embodiments include, but are not limited to, poly(glycolic) acid (PGA), poly(lactide) acid (PLA), poly(lactic-co-glycolic) acid (PLGA), poly(dioxanone), poly(trimethylene carbonate) copolymers, and poly (ε-caprolactone) (PLC) homopolymers and copolymers, polyanhydrides, polyorthoesters, polyphosphazenes, and other biodegradable polymers. Cellulose materials are also considered suitable, such as, carboxymethyl cellulose (CMC) and Hydroxylpropyl methyl cellulose (HMC). Extracellular matrix materials with collagen, including small intestine submucosa (SIS) or other known collagen materials may be used, which are described in more detail below. Additional materials known to be biodegradable may be listed below in the list of biocompatible materials.

For medical devices in which the layers are formed of different biodegradable materials, a skilled artisan will be able to select two or three suitable biodegradable materials for use in a particular medical device based on various considerations. For example, if a desired directional release of the bioactive in the medical device is desired, a first biodegradable material that degrades relatively quickly in vivo may be used as the outer layer in the medical device, and a second biodegradable material that degrades relatively slowly in vivo may be used as the first and/or second inner layers in the medical device. In a medical device having such layers, the outer layer will degrade relatively quickly, resulting in release of the bioactive beads to the surrounding tissue at the point of treatment, while the first inner layer will degrade relatively slowly, effectively providing a barrier to release of the second layer of bioactive beads.

The medical devices may have any suitable size, shape and configuration. Likewise, each of the layers in a particular medical device may have any suitable size, shape and configuration. A skilled artisan will be able to select an overall size, shape and configuration for a medical device according to a particular embodiment based on various considerations, including spatial properties of the point of treatment at which the medical device is intended to be used. Similarly, a skilled artisan will be able to select an overall size, shape and configuration for each of the layers in a medical device according to a particular embodiment based on various considerations, including the desired overall size, shape and configuration of the medical device and the size, shape and configuration of the other layer in the medical device.

Each of the layers in a medical device according to a particular embodiment may have any suitable thickness extending between the first and second surfaces, such as an outer surface 405A and/or an inner surface 405B of the support structure 405 for the layers in the medical device. Furthermore, in a particular medical device, the layers can have the same or different thicknesses. A skilled artisan will be able to select suitable thicknesses for each of the layers in a medical device according to a particular embodiment based on various considerations, including the bioactive or bioactives in the beads in the medical device, the nature of the material of which the layer is made, any desired release profile and/or dynamics for the bioactive or bioactives, and the spatial configuration of the point of treatment at which the medical device is intended to be used. Indeed, each layer in a medical device according to a particular embodiment may comprise a sheet, a film, or a thin film.

With reference to FIG. 5, the bioactive beads (for example bioactive first bead 420 as representative of the other beads disclosed herein) may include a biocompatible body 470. The beads may be sized in the nanometer range to refer to the beads as nanoparticles, such as, for example, nanospheres. The body 470 may be cylindrical, spherical (as shown), elliptical, or irregularly shaped. The body 470 may be formed of a biocompatible material listed below, such as biostable materials selected from nonlimiting examples of polytetrafluoroethylenes (PTFE), polyesters, polymethyl-methacrylates, silicones, poly-L-lactic acid, polyglycolic acid, polycaprolactone, polyorthoesters, polyanhydrides, magnesium alloy, or Fe35Mn alloys. In one example, the body 470 of at least some of the beads may be a porous body having a plurality of pores 475. In another example, the body 470 of at least some of the beads may be a nonporous body, without any pores, with the surface of the body 470 configured to receive a bead coating of a bioactive or other agent as described below. The term “pore” is used to refer to any shaped reservoir including, but not limited to, wells, channels, grooves, and throughbores. In one example, any one of the beads of any of the layers may be formed from any one of the biodegradable materials described herein.

FIGS. 6A-6C depict magnified partial sectional views of one of the pores 475 formed in the body 470. The pores 475 are configured to receive a bioactive agent 478. In one example, as shown in FIG. 6A, each of the pores 475 may be capped with a cap coating 480 for controlling the release of the bioactive agent from the pores, with the bead coated omitted. Alternatively, as shown in FIG. 6B, the body 470 of the porous body, including pores 475, may be coated with a bead coating 482 for controlling the release of the bioactive agent from the pores, with the cap coating omitted. The cap coating and/or bead coating material may depend on the property of bead carrying bioactive agent, such as but not limited to, hydrophilic, hydrophobic, charge, or inert. The cap coating and/or bead coating material may depend on the property of the concentration of bioactive agent. Example of material used in the cap coating 480 and/or the bead coating 482 may include, but not limited to, PLA, PGA, PLLA, fluorinated polymethacrylate PFM-P75, or other biodegradable materials disclosed herein. In another example, as shown in FIG. 6C, each of the pores 475 may be capped with the cap coating 480 for controlling the release of the bioactive agent from the pores, and the porous body of may be coated with the bead coating 482.

In one embodiment, at least one of the layer of first beads 420 comprises a first bioactive agent and at least another of the beads comprises a second, different bioactive agent. Alternatively, some of the pores 475 of the first beads 420 include a first bioactive agent, and the remaining of the pores 475 of the same bead include a second, different bioactive agent. Alternatively, the pores 475 of the first beads 420 may include a first bioactive agent, and the cap coating 480 and/or the bead coating 482 includes a second, different bioactive agent. Alternatively, the pores 475 of the beads 420 may include a bioactive agent, and cap coating 480 and/or the bead coating 482 includes a biodegradable material. Alternatively, the pores 475 of the first beads 420 may include a bioactive agent, and one of the cap coating 480 or the bead coating 482 includes a biodegradable material, and the other of the cap coating 480 or the bead coating 482 includes a second bioactive agent. The cap coating and/or bead coating may include biodegradable materials such as ones described herein.

The layers of beads 420, 440, 460 may include any number of bioactive beads. A skilled artisan will be able to select the number based, for example on the size of the beads, the bioactive agent release kinetics desired and provided by each bead, the area over which the bioactive agent is to be locally released, and other factors. For example, one of the first or second layers of beads 420 or 440 may have a greater number of beads than the other of the first or second layer of beads 420 or 440.

As described below, the medical device may be used as a bioactive delivery device having the same or different therapeutic effects after sequential periods of time. Each of the layers of beads 420, 440, 460 may be formed from the same bioactive agents or different bioactive agents. The bioactive agents in one of the layers of beads may be formed from the same substance having different characteristics such as dosages. Alternatively, at least one of the layers of beads 420, 440, 460 may include a plurality of bioactive agents, with the beads including two bioactive agents and/or with beads comprising first beads including a first agent and second beads including a second agent. Third or more bioactive agents may be included in similar fashion.

In one example application, a medical device may be configured for sequentially controlling the release of a first bioactive agent and a second bioactive agent for medical treatment of aortic aneurysm. The coating 400 is disposed on and at least partially covering the support structure 405. The coating 400 includes the outer layer 410, the inner layer 430 coaxially disposed inward relative to the outer layer 410, and the first layer of a plurality of first beads 420 disposed between the layers 410, 430. Each of the first beads 420 includes the biocompatible porous body 470 having the plurality of pores 475. In one example, each of the first beads 420 includes the bead coating 482 operable for binding to a wall of the body vessel. A therapeutically effective amount of the first bioactive agent is disposed within the pores of the first beads 420. A second layer of the plurality of second beads 440 is disposed between the support structure 405 and the inner layer 430. Each of the second beads 440 includes the biocompatible porous body 470 having the plurality of pores 475. A therapeutically effective amount of a second bioactive agent is disposed within the pores of the second beads 440. The second bioactive agent is different from the first bioactive agent. The first beads 420 are releasable in response to at least partial biodegradation of the outer layer 410. The second beads 440 are releasable after the first beads 420 in response to at least partial biodegradation of the inner layer 430.

In one example, the outer layer 410 may be operable for degradation within up to four to eight hours after implantation of the medical device. The first beads 420 are released from the coating after the degradation. The first beads will be released toward the body vessel wall. The bead coating 482 of the first beads may facilitate binding to the body vessel wall. For example, the bead coating may include a tissue-targeting substance. After the bead coating biodegrades, such as quickly less than one hour, upon binding to the body vessel wall, the first bioactive agent may include thrombolytics, such as, for example, urokinase, alteplase, reteplase, streptokinase, tenecteplase, staphylokinase, herapin, or any of the bioactive agents described herein, operable to reduce blood clotting. The first bioactive agent may include an anti-plaque agent operable to inhibit plaque formation and/or dissolve plaque formed within the blood vessel. The first bioactive agent may include a coagulant may be provided to reduce the incidence of Type II endoleaks (blood from into the aneurysmal sack from branch arteries). The first beads may include at least one of or two bioactive agents, such as the thrombolytics and anti-plaque agent in the configurations described above. The biodegradable inner layer 430 may be operable for degradation within up to 2 to 3 weeks after implantation of the medical device. The second beads may include pro-coagulant agent, such as, for example, thrombin, collagen,Ca²⁺-ionophore A23187, or any of the bioactive agents described herein, operable to increase blood clotting, for example, in response to an endoleak. The second beads may promote fibrosis within the aneurysmal sack to fill the space. The biostable second beads may also contribute to blood clotting and resolving an endoleak. When the medical device includes a second biodegradable inner layer, the second biodegradable inner layer 450 is operable for degradation after the degradation of the biodegradable inner liner 430, and when interfacing with sufficient amount of fresh blood caused by an endoleak. The third layer of beads 460 may include pro-coagulants operable to increase blood clotting, for example, in response to the endoleak. The biodegradable outer layer is configured for faster and/or earlier degradation in vivo than the biodegradable inner layer.

In one example, the medical device includes the outer layer 410, and a plurality of bioactive first beads 420 disposed between the outer layer 410 and the support structure 405, with the other layers in FIG. 4 omitted. The first beads 420 may include the biostable porous body and the bead coating including the tissue targeting substance operable for binding to a wall of the body. The therapeutically effective amount of the first bioactive agent is disposed within the pores of the biostable porous body of the first beads. The beads are releasable in response to at least partial biodegradation of the biodegradable layer, and the first bioactive agent is releasable in response to at least partial biodegradation of the bead coating.

The tissue-targeting substance used in the bead coating 482, such as but not limited to, lipoproteins, glycoproteins, asialoglycoproteins, transferrin, toxins, carbohydrates, cell surface receptor ligands, antibodies, and homing peptides. Beads with tissue-targeting substances may need to be added just prior to implantation. A needle or bead injector device may be connected to a source of freeze dried bioactive beads having the tissue-targeting substance and configured for delivery within the spacing between biodegradable layers. The tissue-targeting substance may need freeze dried for preservation and effectiveness once the medical device is implanted. A patch may be used to seal the locations of penetrations used to deliver the beads.

FIG. 7 illustrates an example of a different coating configuration 500 for a medical device, such as any one of the stent grafts disclosed herein. The coating 500 includes many of the same components as shown in the coating 400, with the addition of a nonwoven matrix layers 502, 504, 506. The layers 410, 430, and/or 450 are shown disposed along the nonwoven matrix layers. As shown, the layers of beads 420, 440, and/or 460 may be distributed within the nonwoven matrix layers 502, 504, 506. The beads may reside within the openings formed by the network of fibers of the nonwoven matrix. For example, one nonwoven matrix layer may be disposed along the support structure. The corresponding beads may be distributed within the openings of the nonwoven matrix layer. Any one of the biodegradable layers may be applied along the nonwoven matrix layer or bead layer to capture the beads within the layers, such as, but not limited to, by spraying, brushing, or dipping. Subsequent nonwoven matrix layers, bead layers, and biodegradable layers may be applied, as described herein.

Coatings or graft bodies operable for increasing porosity over time of implantation may be beneficial. The initial lack of porosity may inhibit blood or fluid into the medical device. Increasing porosity may allow the incorporation of the outer coating or graft body into the surface of the body vessel wall with infiltration of fibroblasts that may further inhibit migration of the medical device due to the body's integration. Extracellular matrix growth may be possible into the inner coating disposed along a luminal surface of the medical device with eventual endothelialization of the inner coating, which may reduce late thrombosis formation. In one example, solutions for forming first fibers in the nonwoven matrix layers may include biocompatible and biodegradable materials, and solutions for forming second fibers in the nonwoven matrix layers may include biocompatible and biostable (or lack biodegradation properties) materials, resulting in a matrix layer having an increasing porosity as the biodegradable fibers degrade and the biostable fibers remain. When placed in an in vivo environment, the biodegradable fibers begin to degrade to leave voids that receive the patient's own native collagen. The biostable fibers remains and functions as a scaffold for infiltrating cells and collagen as well as providing a long-term physical barrier between the blood flow and the injured and/or weakened vessel wall.

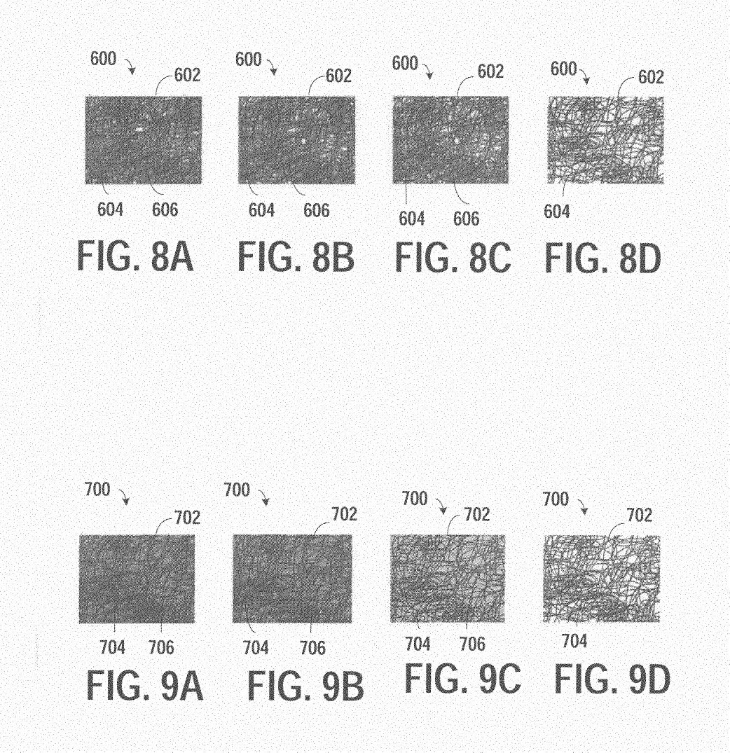

FIGS. 8A-8D depict an example of a different coating configuration 600 for a medical device, such as any one of the stent grafts disclosed herein, degrading at four distinctive and sequential times at or after implantation of the medical device into the body vessel. FIG. 8A depicts the coating 600 closer to initial implantation, whereas FIG. 8D depicts the coating 600 after the greater period of time from initial implantation. The coating 600 includes a nonwoven matrix layer 602 that is disposed along the support structure. In one example, the nonwoven matrix layer 602 includes biostable fibers 604 and biodegradable fibers 606 entangled with the biostable fibers 604 in a network of fibers. The biostable fibers 604 may include any of one of the biocompatible materials described herein. The biodegradable fibers 606 may include any of one of the biodegradable materials described herein. In one example, the solution for forming the biodegradable fibers may also include a bioactive agent, such as any one of the bioactive agents disclosed herein. In this case, the bioactive agent may elute during the degradation process of the fibers. FIG. 8D depicts the total biodegradation of the biodegradable fibers, leaving only the biostable fibers 604. In another example, the nonwoven matrix layer 602 may include additional layers of a binding layer and a bioactive agent layer. A plurality of bioactive agent layers and binding layers may be positioned stacked on top of each other. Binding layer is operable to adhere the bioactive layer to the nonwoven matrix. In another example, beads, such as described previously, may be distributed within the network of fibers of the nonwoven matrix layer.

The bioactive agents described herein may be bonded to the nonwoven matrix layer, either directly via a covalent bond or via a linker molecule, which covalently links the bioactive agent and the coating layer. Alternatively, the bioactive agent may be bound to the coating layer by ionic interactions via the binding layer including cationic polymer coatings with anionic functionality on bioactive agent or alternatively anionic polymer coatings with cationic functionality on the bioactive agent. Hydrophobic interactions may also be used to bind the bioactive agent to the binding layer including a hydrophobic portion. The bioactive agent may be modified to include a hydrophobic moiety such as a carbon-based moiety, silicon-carbon based moiety or other such hydrophobic moiety. Alternatively, the hydrogen bonding interactions may be used to bind the bioactive agent to the coating layer.

FIGS. 9A-9D depict an example of a different coating configuration 700 for a medical device, such as any one of the stent grafts disclosed herein, degrading at four distinctive times at or after implantation into the body vessel. FIG. 9A depicts the coating 700 closer to initial implantation, whereas FIG. 9D depicts the coating 700 after the greater period of time from initial implantation. The coating 700 includes a nonwoven matrix layer 702 that is disposed along the support structure. In one example, the nonwoven matrix layer 702 includes biostable fibers 704 entangled with one another in a network of fibers. The biostable fibers 704 may include any of one of the biocompatible materials described herein. A biodegradable layer 706 is applied to the nonwoven matrix layer 702. The biodegradable layer 706 may be applied by various techniques, including but not limited to, by spraying, brushing, or dipping to the layer 702 or configured as a sheet heat bonded to the layer 702. The biodegradable layer solution fills the pores formed by the network of fibers. Solvent may be used in the biodegradable layer solution is allowed to evaporate or is removed through the application of a vacuum, leaving behind the biodegradable layer. In one example, the solution for forming the biodegradable layer 706 may also include a bioactive agent, such as any one of the bioactive agents disclosed herein. In this case, the bioactive agent may elute during the degradation process of the layer. FIG. 9D depicts the total biodegradation of the biodegradable layer, leaving only the biostable fibers 704. In another example, the nonwoven matrix layer 702 may include additional layers of a binding layer and a bioactive agent layer. A plurality of bioactive agent layers and binding layers may be positioned stacked on top of each other. Binding layer is operable to adhere the bioactive layer to the nonwoven matrix. In another example, beads, such as described previously, may be distributed within the network of fibers of the nonwoven matrix layer.

The nonwoven matrix layers described herein may be formed from the same materials or different materials. The nonwoven matrix layers may be formed from the same materials having different characteristics such as wall thickness, porosity, fiber thickness, or fiber makeup, or different materials having different characteristics such as wall thickness, porosity, fiber thickness, or fiber makeup.

The nonwoven matrix layers may be a sheet of fabric created by fiber or filament entanglement accomplished by mechanical, thermal, and/or chemical methods. In one example, the nonwoven layer is formed by melt spinning process such as, for example, melt blowing. In another example, the nonwoven matrix layer may be formed using an electrospinning process and may include a plurality of continuous electrospun fibers. Electrospinning process uses an electrically charged solution that is driven from a source to a target with an electrical field. For example, a solution is driven from an orifice, such as a needle. A voltage is applied to the orifice resulting in a charged solution jet or stream from the orifice to the target. The jet forms a conical shape, termed a Taylor cone, as it travels from the orifice. As the distance from the orifice increases, the cone becomes stretched until the jet splits or splays into many fibers prior to reaching the target. The fibers are extremely thin, typically in the nanometer range. The collection of fibers on the target forms a thin mesh layer of fibrous material.

It may be expected that a majority of the electrospun fibers of the graft material may be continuous or discontinuous. The network of electrospun fibers may define openings along the layer. Opening may also be formed by laser cutting, mechanical punching, or other know cutting means for precisely controlling the size and/or shape of each opening formed by electrospinning. Additionally, the openings of the electrospun-patterned coating may be homogeneous. Forming openings by laser marking or mechanical punching in a subsequent processing step after formation of the coating may sever individual fibers of the graft material surrounding the openings, resulting in melted or frayed fiber ends. The openings also may be uniform. This may provide a coating having a precisely controlled porosity.

For example, the solutions for forming the fibers in the nonwoven matrix layers may be biocompatible and biostable (or lack biodegradation properties), resulting in a biostable matrix layer. Alternatively, the solutions for forming the fibers in the nonwoven matrix layers may be biocompatible and biodegradable, resulting in a biodegradable matrix layer.

Solutions for use in the electrospinning process may include any suitable liquids containing materials to be electrospun. For example, solutions may include, but are not limited to, suspensions, emulsions, melts, and hydrated gels containing the materials, substances, or compounds to be electrospun. Solutions also may include solvents or other liquids or carrier molecules. Appropriate materials for electrospinning may include any compound, molecule, substance, or group or combination thereof that may form any type of structure or group of structures during or after electrospinning. For example, materials may include natural materials, synthetic materials, or combinations thereof. Naturally-occurring organic materials may include any substances naturally found in the body of plants or other organisms, regardless of whether those materials have or can be produced or altered synthetically. Synthetic materials may include any materials prepared through any method of artificial synthesis, processing, or manufacture. In one example, the materials may be biocompatible materials. Such materials may include, for example, any materials that may be used to form an aspect of the coating and/or a graft material of the medical device as described above.

Solutions may include any solvent that allows delivery of the material or substance to the orifice, tip of a syringe, or other site from which the material may be electrospun. The solvent may be used for dissolving or suspending the material or the substance to be electrospun. For example, solvents for use in electrospinning may create a mixture with collagen and/or other materials to be electrospun, to enable electrospinning of such materials. Suitable solvents may include, for example, trifluoroacetic acid, dichloromethane, dimethylacetamide (DMAc), or any other suitable solvent. Examples of the use of solvents, proteins, and other agents is described in U.S. Pat. No. 9,175,427, granted Nov. 3, 2015, entitled “Electrospun Patterned Stent Graft Covering,” assigned to Cook Medical Technologies LLC, which is incorporated herein in its entirety by reference.

Solutions for electrospinning the biodegradable fibers or for dipping or applying the biodegradable coating also may include one or more bioactive agents. A therapeutically effective amount of a bioactive agent by be incorporated into the fibers and/or coating for implantation within a patient. The bioactive agent may be selected to perform a desired function upon implantation.

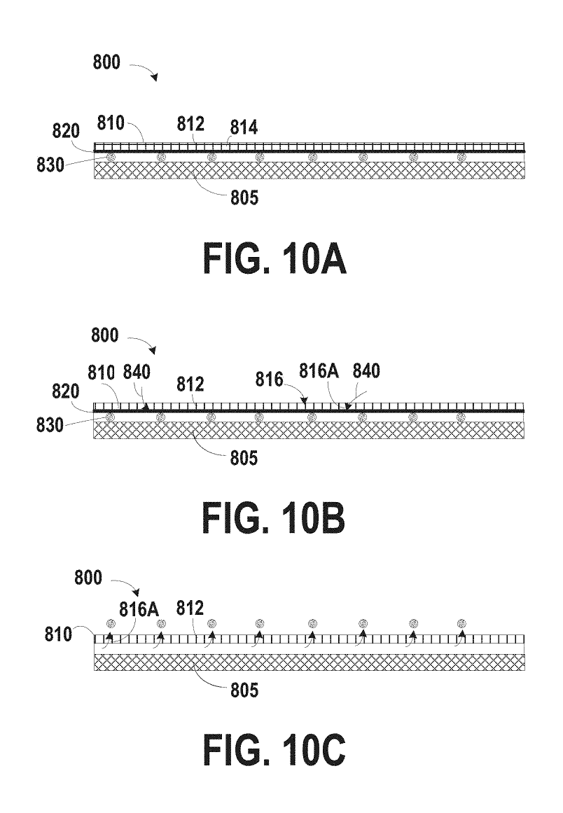

FIGS. 10A-10C depict another example of a different coating configuration 800 for a medical device, such as any one of the stent grafts disclosed herein. FIG. 10A depicts the coating 800 closer to initial implantation, whereas FIG. 10C depicts the coating 800 after the greater period of time from initial implantation. The coating 800 includes an outer layer 810, an intermediate biodegradable layer 820, and a plurality of bioactive beads 830 forming a beaded layer disposed between the biodegradable layer 820 and the support structure 805, all of which are coaxially disposed relative to one another. The outer layer 810 may comprise a woven or nonwoven matrix layer is disposed along the support structure. In one example, the outer layer includes biostable fibers 812 and biodegradable fibers 814. In the case of woven, the biostable fibers 812 (illustrated schematically as vertical lines) and the biodegradable fibers 814 (illustrated schematically as horizontal lines) are woven together or other braided or entangled in a regular textile pattern. In the case of nonwoven, the biostable fibers 812 and the biodegradable fibers 814 are entangled in an irregular pattern such as already described. The biostable fibers 812 and the biodegradable fibers 814 may include any of one of the respective biocompatible materials described herein. The biodegradable layer 820 is applied along the inside of the outer layer 810. The biodegradable layer 820 may be applied by various techniques, including but not limited to, by spraying, brushing, or dipping to the layer 810 or configured as a sheet heat bonded to the layer 810. The biodegradable layer 820 may be in a solution form to fill the pores formed by the network of fibers. Solvent may be used in the biodegradable layer solution is allowed to evaporate or is removed through the application of a vacuum, leaving behind the biodegradable layer. The bioactive beads 830, such as described above, are disposed between the biodegradable layer 820 and the support structure 805. When the support structure 805 comprises a graft material, such graft material may define pores (not shown) either by the design of the graft material or by the graft be a woven or nonwoven construct. It is contemplated that such graft material pores may be sized to receive the indwelling bioactive beads 830 and the biodegradable layer 820 may be disposed directly on the support structure 805 or substantially closer than what appears schematically in the figures.

The beads 830 with the bioactive agent are releasable from the coating in response to biodegradation of the biodegradable material fibers of the outer layer 810 at a first time after implantation of the device, and at least partial biodegradation of the biodegradable layer 820 at a second time subsequent to the first time after implantation. FIG. 10B depicts the configuration of the coating 800 at a period of time after implantation. The biodegradable fibers 814 are now dissolved, leaving the biostable fibers 812 alone to define the outer layer 810. The biostable fibers 812 are now arranged relative to one another to define pores 816 into the outer layer 810. At least a partial number of the pores (now 816A) extend through the outer layer 810 and open to the biodegradable layer 820. The pores 816A opened to the layer 820 allow body fluid and blood (shown by arrows 840) to interface with the biodegradable layer 820 to initiate the biodegradation of the biodegradable layer 820.

FIG. 10C depicts another configuration of the coating 800, with the total biodegradation of the biodegradable layer 820, leaving only the porous outer layer 810. The period of time for total biodegradation of the layer 820 would be subsequent to the period of time associated with the configuration shown in FIG. 10B. As shown by the arrows, the pores 816A are sized and shaped to allow the passage of the bioactive beads 830 through the outer layer 810 and release into the body for therapeutically treating the body such as the aneurysm sack. The bioactive beads are shown schematically larger than what the beads would be at a microscopic level.

For example, the bioactive agent may be selected to treat indications such as atherosclerosis, renal dialysis fistulae stenosis, or vascular graft stenosis. A coating of a graft material including a bioactive agent may be useful when performing procedures such as coronary artery angioplasty, renal artery angioplasty, or carotid artery surgery. Also for example, a bioactive agent such as a growth factor may be selected to promote ingrowth of tissue from the interior wall of a body vessel. An anti-angiogenic or antineoplastic bioactive agent such as paclitaxel, sirolimus or a rapamycin analog, such as zotarolimus, everolimus, biolimus, or a metalloproteinase inhibitor such as batimastaat may be included to mitigate or prevent undesired conditions in the vessel wall, such as restenosis. Many other types of bioactive agents also may be included in the solution.

Just some examples of the large range of bioactive materials which can be applied to the medical device for treating targeted diseases or issues include but are not limited to: paclitaxel, heparin, azathioprine or azathioprine sodium; basiliximab; cyclosporin or cyclosporine (cyclosporin A); daclizumab (dacliximab); glatiramer or glatiramer acetate; muromonab-CD3; mycophenolate, mycophenolate mofetil (MMF), mycophenolate morpholinoethyl or mycophenolic acid; tacrolimus (FK506), anhydrous tacrolimus or tacrolimus monohydrate; sirolimus; interferon alfa-2a, recombinant (rIFN-A or IFLrA); antilymphocyte immunoglobulin (ALG), antithymocyte immunoglobulin (ATG), antilymphocyte serum, antithymocyte serum, lymphocytic antiserum or thymitic antiserum; brequinar or brequinar sodium; cyclophosphamide, cyclophosphamide monohydrate or anhydrous cyclophosphamide; dactinomycin, actinomycin C, actinomycin D or meractinomycin; daunorubicin, daunorubicin hydrochloride, daunomycin hydrochloride or rubidomycin hydrochloride; doxorubicin, doxorubicin hydrochloride, adriamycin or adriamycin hydrochloride; fluorouracil; gusperimus or gusperimus hydrochloride; inolimomab; leflunomide; mercaptopurine, mercaptopurine monohydrate, purinethiol or anhydrous mercaptopurine; methotrexate, methotrexate sodium, methotrexate disodium, alpha-methopterin or amethopterin; mustine, mustine hydrochloride, chlormethine hydrochloride, chlorethazine hydrochloride, mechlorethamine hydrochloride or nitrogen mustard (mustine); mizoribine; vinblastine, vinblastine sulfate or vincaleukoblastine sulphate; a pharmacologically or physiologically acceptable salt of any of the foregoing; or a pharmacologically or physiologically acceptable mixture of any two or more of the foregoing. These bioactive agents have effects known in the art including as thrombolytics, vasodilators, antihypertensive agents, antimicrobials or antibiotics, antimitotics, antiproliferatives, antisecretory agents, non-steroidal anti-inflammatory drugs, immunosuppressive agents, growth factors and growth factor antagonists, antitumor and/or chemotherapeutic agents, antipolymerases, antiviral agents, photodynamic therapy agents, antibody targeted therapy agents, prodrugs, sex hormones, free radical scavengers, antioxidants, biologic agents, radiotherapeutic agents, radiopaque agents and radiolabelled agents.

The beads may include a single bioactive, two bioactives, or a plurality of bioactives. When more than one bioactive is used, the bioactives may be mixed prior to being disposed on another component of the medical device according to an embodiment, or may be separately disposed on components of a medical device according to an embodiment. For example, a layer or a solution containing a first bioactive can be applied to the beads in the first bead layer, a biodegradable first fiber, and/or biodegradable first layer of a medical device, as described above, and a layer or solution containing a second bioactive can be applied to the beads in the second bead layer, second beads in the first layer, first beads in the first layer, a biodegradable second or first fiber, and/or biodegradable second or first layer of the medical device.

The term “graft” describes an object, device, or structure that is joined or that is capable of being joined to a body part to enhance, repair, or replace a portion or a function of that body part. Grafts that can be used to repair body vessels include, for example, films, coatings, or sheets of material that are formed or adapted to conform to the body vessel that is being enhanced, repaired, or replaced. The graft material may include a biocompatible synthetic or biomaterial. Examples of suitable synthetic materials include fabrics, woven and nonwoven materials, and porous and nonporous sheet materials. Other synthetic graft materials include biocompatible materials such as polyester, polytetrafluoroethylene (PTFE), polyurethane, and the like. Examples of suitable biocompatible materials include, for example, pericardial tissue and extracellular matrix materials such as SIS.

In one example, the solution may include synthetic materials, such as biocompatible synthetic materials. Synthetic materials may include polymers such as, for example, poly(urethanes), poly(siloxanes) or silicones, poly(ethylene), poly(vinyl pyrrolidone), poly(2-hydroxy ethyl methacrylate), poly(N-vinyl pyrrolidone), poly(methyl methacrylate), poly(vinyl alcohol), poly(acrylic acid), polyacrylamide, poly(ethylene-co-vinyl acetate), poly(ethylene glycol), poly(methacrylic acid), polylactides (PLA), polyglycolides (PGA), poly(lactide-co-glycolid-es) (PLGA), polyanhydrides, polyorthoesters or any other similar synthetic polymers that may be developed that are biocompatible. Biocompatible synthetic polymers also may include copolymers, blends, or any other combinations of the forgoing materials either together or with other polymers generally. The use of these polymers will depend on given applications and specifications required. Suitable polymer material may include, for example, polyester such as DACRON™, polyetherurethanes such as THORALON® from Thoratec Corporation (Pleasanton, Calif.), or polyethylene terephthalate (PET).

In addition, materials that are not inherently biocompatible may be subjected to surface modifications in order to render the materials biocompatible. Examples of surface modifications include graft polymerization of biocompatible polymers from the material surface, coating of the surface with a crosslinked biocompatible polymer, chemical modification with biocompatible functional groups, and immobilization of a compatibilizing agent such as heparin or other substances. Thus, any polymer that may be formed into a porous sheet can be used to make a graft material, provided the final porous material is biocompatible. Polymers that can be formed into a porous sheet include polyolefins, polyacrylonitrile, nylons, polyaramids and polysulfones, in addition to polyesters, fluorinated polymers, polysiloxanes and polyurethanes as listed above. Preferably, the porous sheet is made of one or more polymers that do not require treatment or modification to be biocompatible.

The graft material, the coating, or one class of materials for electrospinning may also include extracellular matrix materials. The “extracellular matrix” is typically a collagen-rich substance that is found in between cells in animal tissue and serves as a structural element in tissues. Such an extracellular matrix is preferably a complex mixture of polysaccharides and proteins secreted by cells. The extracellular matrix can be isolated and treated in a variety of ways. Following isolation and treatment, it is referred to as an “extracellular matrix material,” or ECMM. ECMMs may be isolated from submucosa (including small intestine submucosa), stomach submucosa, urinary bladder submucosa, tissue mucosa, renal capsule, dura mater, liver basement membrane, pericardium or other tissues.

The rate of biodegradation of the biodegradable layers may be selected based on materials and the layer thickness. In the case of nonwoven matrix layers, the density of fibers, number of fibers, fiber thickness, fiber material may also be selected to control the rate of biodegradation. For example, within the initial 4 to 8 hours post-implantation, a coagulant may be desired to reduce the incidence of Type II endoleak (blood flows into the aneurysmal sack from branch arteries). This may release from the outer most layer of the graft into the aneurysmal sack or space. The coagulant contained within a fast absorbing or dissolving bead. Long-term, for example, 2-3 weeks post-implantation, a second type of bead is released, located deeper within the graft material, which results in fibrosis within the aneurysmal sack to fill the space.

The stent or support frame structures may be any device or structure that provides or is configured to provide rigidity, expansion force, or support to a body part, for example, a diseased, damaged, or otherwise compromised body lumen. Such stent structure may include any suitable biocompatible material, including, but not limited to fabrics, metals, plastics, and the like. Examples of suitable materials include metals such as stainless steel and nitinol, and plastics such as polyethylene terephthalate (“PET”), polytetrafluoroethylene (“PTFE”) and polyurethane. The stent structure may be “expandable,” that is, it may be capable of being expanded to a larger-dimension configuration. The stent structure may expand by virtue of its own resilience (i.e., self-expanding), upon the application of an external force (i.e., balloon-expandable), or by a combination of both. In one example, the stent structure may have one or more self-expanding portions and one or more balloon-expandable portions. The stent struts that are interconnected to one another represents specific configurations of a wire member that comprises a basic structural component of the stent. As used herein, the term “wire” refers to any filamentary member, including, but not limited to, drawn wire and filaments that have been laser cut from a cannula. For example, the stent architecture with the intricate mating elements that form the interlocking joints may lend itself to being manufacture from a metal cannula laser cut to the desired pattern as described.

The graft material and/or coating may be attached to a stent structure by various means. The graft material may be attached to the stent by stitching, for example by using a monofilament or braded suture material. The graft material also may be affixed to the stent by dipping the stent in a liquefied polymer and allowing the polymer to solidify into a film. The liquefied polymer may be a molten polymer or a polymer or pre-polymer before curing or cross-linking occurs.

The shape, size, and dimensions of the stent structure may vary. The size of these components and the overall stent structure is determined primarily by the diameter of the vessel lumen at the intended implant site, as well as the desired length of the overall stent device. The stent structure and/or ring structures may have a common cross-sectional area along the body or may vary to have different cross-sectional areas.

The medical devices may be made by performance of the methods described herein. One example medical device comprises a medical device made by a method of making a medical device for delivering of bioactives. The method may include disposing a layer of bioactive beads along a support structure, disposing a biodegradable layer on the beads; disposing another layer of bioactive beads; and securing the first and second biodegradable layers to each other to the support structure. The method can optionally include forming a triple laminate structure on the periphery of the first and second biodegradable layers, including another biodegradable layer and bead layer. In one example, a portion of the beads are disposed in a controlled environment, such as freeze drying. A pocket is formed between the biodegradable layers, such as with a spacer material, and the freeze dried beads are delivered into the pocket, prior to implantation.

Methods of using any one of the medical devices described herein, such as by placing a medical device described herein into a body at a point of treatment, such as a point of treatment in an aortic system having an aneurysm. Also, methods of using the medical devices described herein in combination with another medical device, such as by placing a medical device described herein as a branch connecting stent within a fenestrated stent graft, and placing the medical devices together into a body at a point of treatment, such as a point of treatment in the aortic system. The medical device may be delivered with suitable techniques, depending on the type of medical device. In one example, access to the body may be attained by inserting an access device, such as an introducer sheath, into the body passageway. One typical procedure for inserting the introducer sheath over an inserted wire guide using the well-known Seldinger percutaneous entry technique. The medical device may be delivered with a stent deployment system using the introducer sheath, and advanced to the treatment site, such as the aneurysm shown in FIG. 2, typically using visual techniques such as fluoroscopy. The medical device may be radially compressed to a lower profile for delivery. An outer sheath is moved relative to the medical device to allow for radial expansion within the body. Trigger wires may be provided and activated for selective expansion of the medical device. Once implanted, the system may be removed from the body. After implantation, layers of the coating begin to biodegrade, and the bioactive agents begin eluting into the body in a controlled manner such as described above.

To clarify the use of and to hereby provide notice to the public, the phrases “at least one of <A>, <B>, . . . and <N>” or “at least one of <A>, <B>, . . . <N>, or combinations thereof” or “<A>, <B>, . . . and/or <N>” are defined by the Applicant in the broadest sense, superseding any other implied definitions hereinbefore or hereinafter unless expressly asserted by the Applicant to the contrary, to mean one or more elements selected from the group comprising A, B, . . . and N. In other words, the phrases mean any combination of one or more of the elements A, B, . . . or N including any one element alone or the one element in combination with one or more of the other elements which may also include, in combination, additional elements not listed.

While various embodiments of the invention have been described, the invention is not to be restricted except in light of the attached claims and their equivalents. Moreover, the advantages described herein are not necessarily the only advantages of the invention and it is not necessarily expected that every embodiment of the invention will achieve all of the advantages described.

The subject-matter of the disclosure may also relate, among others, to the following aspects:

1. A medical device for controlling a release of bioactive agents, the medical device comprising: a support structure configured and sized to be used within a body of a human or an animal; a coating at least partially covering an exterior of the support structure, the coating comprising: a biodegradable outer layer; a biodegradable inner layer coaxially disposed inward relative to the biodegradable outer layer; a plurality of first beads disposed between the outer and inner layers, each of the first beads comprising a biocompatible porous body having a plurality of pores, each of the first beads including a bead coating operable for binding to a wall of the body, wherein a therapeutically effective amount of a first bioactive agent is disposed within the pores of the first beads; a plurality of second beads disposed between the support structure and the inner layer, each of the second beads comprising a biocompatible porous body having a plurality of pores, wherein a therapeutically effective amount of a second bioactive agent is disposed within the pores of the second beads, wherein the first beads are releasable in response to at least partial biodegradation of the outer layer, and the second beads are releasable after the first beads in response to at least partial biodegradation of the inner layer.

2. The device of aspect 1, wherein the support structure includes a graft material.

3. The device of any one of aspects 1-2, wherein the biodegradable outer layer is configured to degrade faster in vivo than the biodegradable inner layer.

4. The device any one of aspects 1-3, wherein at least one of the first beads and the second beads include a biodegradable cap coating disposed within the pores.

5. The device of any one of aspects 1-4, wherein the bead coating includes a tissue targeting substance.

6. The device of any one of aspects 1-5, wherein the first bioactive agent includes at least one of thrombolytic and/or an anti-plaque agent.

7. The device of any one of aspects 1-6, wherein the second bioactive agent includes a pro-coagulant agent.

8. The device of any one of aspects 1-7, wherein the coating comprises a nonwoven matrix layer.

9. The device of any one of aspects 1-8, wherein the nonwoven matrix layer is disposed around at least one of the first beads and the second beads.

10. The device of any one of aspects 1-9, furthering comprising a plurality of third beads disposed along at least one of the first beads and the second beads, wherein the third bead includes a therapeutically effective amount of a third bioactive agent.

11. The device of any one of aspects 1-10, wherein at least one the first beads and the second beads includes a therapeutically effective amount of a third bioactive agent.

12. A medical device for controlling a release of a bioactive agent, the medical device comprising: a support structure configured and sized to be used within a body of a human or an animal, the support structure including at least a graft material, and a coating at least partially covering the support structure, the io coating comprising: an outer layer comprising a biodegradable material and a biostable material; a biodegradable layer disposed along an inside of the outer layer; and a plurality of beads disposed between the biodegradable layer and the support structure, each of the beads comprising a biostable porous body, wherein a therapeutically effective amount of a bioactive agent is disposed within a plurality of pores of the biostable porous body, wherein the beads with the bioactive agent are releasable from the coating in response to biodegradation of the biodegradable material of the outer layer at a first time, and at least partial biodegradation of the biodegradable layer at a second time subsequent to the first time.

13. The medical device of aspect 12, wherein the outer layer includes a plurality of biodegradable material fibers and a plurality of biostable material fibers, the biodegradable material fibers and the biostable material fibers are in a woven or nonwoven pattern.

14. The medical device of any one of aspects 12-13, wherein the support structure includes a radially expandable stent frame structure to support the graft material.

15. The medical device of any one of aspects 12-14, wherein the bioactive agent includes at least one of a thrombolytic, an anti-plaque agent, or a pro-coagulant agent.

16. A stent graft for controlling a release of bioactive agents, the stent graft comprising: a radially expandable frame structure and a graft material configured and sized to be used within a body of a human or an animal, and a coating disposed on and at least partially covering an exterior of the graft material, the coating comprising: an outer layer comprising a biodegradable material; a biodegradable inner layer disposed radially inward relative to the outer layer; a plurality of first beads disposed between the outer layer and the biodegradable inner layer, each of the first beads comprising a biostable porous body, wherein a therapeutically effective amount of a first bioactive agent is disposed within the biostable porous body of the first beads; a plurality of second beads disposed between the frame structure and the biodegradable inner layer, each of the second beads comprising a biostable porous body, wherein a therapeutically effective amount of a second bioactive agent is disposed within the biostable porous body of the second beads, wherein the first beads are releasable in response to at least io partial biodegradation of the outer layer, and the second beads are releasable after the first beads in response to at least partial biodegradation of the biodegradable inner layer.

17. The stent graft of aspect 16, wherein the biostable porous body of the first beads includes a bead coating comprising a tissue targeting substance.

18. The stent graft of one of aspects 16-17, wherein the outer layer is operable for biodegradation within up to eight hours after implantation of the stent graft, and the second bioactive agent is different from the first bioactive agent, wherein the biodegradable inner layer is configured to degrade within up to three weeks after implantation of the stent graft.

19. The stent graft of one of aspects 16-18, wherein the first bioactive agent includes at least one of thrombolytic and an anti-plaque agent, and the second bioactive agent includes a pro-coagulant agent.

20. The stent graft of one of aspects 16-19, wherein at least one of the first bioactive beads and the second bioactive beads are included within a nonwoven matrix layer.

We claim:

1. A medical device for controlling a release of bioactive agents, the medical device comprising:
a support structure configured and sized to be used within a body of a human or an animal;
a coating at least partially covering an exterior of the support structure, the coating comprising:
a biodegradable outer layer;
to a biodegradable inner layer coaxially disposed inward relative to the biodegradable outer layer;
a plurality of first beads disposed between the outer and inner layers, each of the first beads comprising a biocompatible porous body having a plurality of pores, each of the first beads including a bead coating operable for binding to a is wall of the body, wherein a therapeutically effective amount of a first bioactive agent is disposed within the pores of the first beads; and
a plurality of second beads disposed between the support structure and the inner layer, each of the second beads comprising a biocompatible porous body having a plurality of pores, wherein a therapeutically effective amount of a second bioactive agent is disposed within the pores of the second beads,
wherein the first beads are releasable in response to at least partial biodegradation of the outer layer, and the second beads are releasable after the first beads in response to at least partial biodegradation of the inner layer.

2. The device of claim 1, wherein the support structure includes a graft material.

3. The device of claim 1, wherein the biodegradable outer layer is configured to biodegrade faster in vivo than the biodegradable inner layer.

4. The device of claim 1, wherein at least one of the first beads and the second beads include a biodegradable cap coating disposed within the pores.

5. The device of claim 1, wherein the bead coating includes a tissue targeting substance.

6. The device of claim 1, wherein the first bioactive agent includes at least one of thrombolytic and an anti-plaque agent.

7. The device of claim 1, wherein the second bioactive agent includes a pro-coagulant agent.

8. The device of claim 1, wherein the coating comprises a nonwoven matrix layer.

9. The device of claim 8, wherein the nonwoven matrix layer is disposed around at least one of the first beads and the second beads.

10. The device of claim 1, furthering comprising a plurality of third beads to disposed along at least one of the first beads and the second beads, wherein the third beads includes a therapeutically effective amount of a third bioactive agent.

11. The device of claim 1, wherein at least one the first beads and the second beads includes a therapeutically effective amount of a third bioactive agent.

12. A medical device for controlling a release of a bioactive agent, the medical is device comprising:
a support structure configured and sized to be used within a body of a human or an animal, the support structure including at least a graft material, and a coating at least partially covering the support structure, the coating comprising:
an outer layer comprising a biodegradable material and a biostable material;
a biodegradable layer disposed along an inside of the outer layer; and
a plurality of beads disposed between the biodegradable layer and the support structure, each of the beads comprising a biostable porous body, wherein a therapeutically effective amount of a bioactive agent is disposed within a plurality of pores of the biostable porous body, wherein the beads with the bioactive agent are releasable from the coating in response to biodegradation of the biodegradable material of the outer layer at a first time, and at least partial biodegradation of the biodegradable layer at a second time subsequent to the first time.

13. The medical device of claim 12, wherein the outer layer includes a plurality of biodegradable material fibers and a plurality of biostable material fibers, the biodegradable material fibers and the biostable material fibers are in a woven or nonwoven pattern.

14. The medical device of claim 13, wherein the support structure includes a radially expandable stent frame structure to support the graft material.

15. The medical device of claim 14, wherein the bioactive agent includes at least one of a thrombolytic, an anti-plaque agent, or a pro-coagulant agent.

16. A stent graft for controlling a release of bioactive agents, the stent graft comprising:
a radially expandable frame structure and a graft material configured and to sized to be used within a body of a human or an animal, and a coating disposed on and at least partially covering an exterior of the graft material, the coating comprising:
an outer layer comprising a biodegradable material;
a biodegradable inner layer disposed radially inward relative to the outer is layer;
a plurality of first beads disposed between the outer layer and the biodegradable inner layer, each of the first beads comprising a biostable porous body, wherein a therapeutically effective amount of a first bioactive agent is disposed within the biostable porous body of the first beads;
a plurality of second beads disposed between the frame structure and the biodegradable inner layer, each of the second beads comprising a biostable porous body, wherein a therapeutically effective amount of a second bioactive agent is disposed within the biostable porous body of the second beads,
wherein the first beads are releasable in response to at least partial biodegradation of the outer layer, and the second beads are releasable after the first beads in response to at least partial biodegradation of the biodegradable inner layer.

17. The stent graft of claim 16, wherein the biostable porous body of the first beads includes a bead coating comprising a tissue targeting substance.

18. The stent graft of claim 17, wherein the outer layer is operable for biodegradation within up to eight hours after implantation of the stent graft, and the second bioactive agent is different from the first bioactive agent, wherein the biodegradable inner layer is configured to degrade within up to three weeks after implantation of the stent graft.

19. The stent graft of claim 18, wherein the first bioactive agent includes at least one of thrombolytic and an anti-plaque agent, and the second bioactive agent includes a pro-coagulant agent.

20. The stent graft of claim 19, wherein at least one of the first bioactive beads and the second bioactive beads are included within a nonwoven matrix layer.
